(19) United States
(12) Patent Application Publication
(10) Pub. No.: US 2004/0063204 A1
(43) Pub. Date: Apr. 1, 2004

(54) Bone marrow cell differentiation

(76) Inventor: Lijun Yang, Gainesville, FL (US)

Correspondence Address: Stanley A. Kim, Ph.D., Esq., Akermann Senterfitt, Suite 400, 222 Lakeview Avenue, West Palm Beach, FL, 33402-3188, US

(21) Appl. No.: 10/640,989

(22) Filed: Aug. 14, 2003

Related U.S. Application Data
(60) Provisional application No. 60/403,579, filed on Aug. 14, 2002.

(51) Int. Cl. C12N 5/08; C12N 5/02; C12N 5/00

(52) U.S. Cl. 435/372; 435/373

(57) ABSTRACT

Human bone marrow-derived stem cells are differentiated into pancreatic endocrine marker-expressing cells in vitro by first culturing human bone marrow mononuclear cells in a tissue culture container to obtain cells that adhere to the container, and continuing to culture the adherent cells until they become morphologically homogenous; second, culturing the morphologically homogeneous cells in a medium containing high glucose levels at least until the cells express detectable levels of glucagon, insulin, and mRNAs encoding insulin, Pdx-1, and NeuroD; and third, culturing the cells in a medium containing low glucose levels, nicotinamide, and exendin 4. Transplantation of pancreatic endocrine marker-expressing cells made in this manner can reduce hyperglycemia in a diabetic animal.

### CROSS-REFERENCE TO RELATED APPLICATION

The present application claims the priority of U.S. provisional patent application serial No. 60/403,579 filed Aug. 14, 2002.

### STATEMENT REGARDING FEDERALLY SPONSPORED RESEARCH

Not applicable.

### FIELD OF THE INVENTION

The invention relates generally to the fields of developmental biology and medicine. More particularly, the invention relates to a method for producing insulin-producing cells from human BM (BM)-derived stem (HBMDS) cells.

### BACKGROUND

Type 1 diabetes is an insulin-dependent, autoimmune disorder characterized by the destruction of insulin-producing beta cells. Current therapeutic options for individuals with type 1 diabetes include insulin replacement therapy, whole pancreas transplantation, and islet cell transplantation. With insulin replacement therapy, it is nearly impossible to achieve euglycemia consistently, and failure to do so results in excursions of blood glucose levels that can lead to acute metabolic complications (e.g., diabetic ketoacidosis and hypoglycemic coma) and potentially long-term complications (e.g., retinopathy, neuropathy, nephropathy, impotence, heart disease, and vascular disease). Pancreas transplantation often establishes an exogenous insulin-free euglycemic state, reduces long-term complications and improves neural and vascular function. Major drawbacks of this procedure include the limited number of human pancreases available for transplantation as well as the requirement for immunosuppressive drugs following transplantation, which may cause alterations in glucose homeostasis and beta cell function. Islet cell transplantation, like whole pancreas transplantation, provides the possibility for internal glycemic control and independence of exogenous insulin. This approach, however, is also hampered by a lack of tissue availability and immunological rejection. One theoretical alternative for islet transplantation involves the use of a renewable source of stem cells capable of self-renewal and differentiation, as well as glucose regulated insulin production. Indeed, the development of a simple, reliable procedure for obtaining autologous stem cells capable of differentiating into functional insulin-producing cells would provide a potentially unlimited source of islet cells for transplantation and alleviate the major limitations of availability and allogeneic rejection.

### SUMMARY

The invention relates to the development of an in vitro method for differentiating HBMDS cells into insulin-producing cells. This method should facilitate the practical implementation of transplantation as a method of treating type 1 diabetes (1) because HBMDS cells can be readily obtained from a subject by non-invasive techniques, and (2) because the use of such cells for autologous transplantation avoids an immune system-mediated reaction that can lead to rejection of transplanted cells.

Accordingly, the invention features an insulin-producing cell isolated from an in vitro culture of BM cells obtained from a human subject. Also within the invention is a method for making a pancreatic marker-expressing cell. This method includes the steps of: (a) obtaining human BM mononuclear cells from a human subject; and (b) culturing the obtained human BM cells under conditions that cause the cultured cells to express detectable levels of glucagon, insulin, and mRNAs encoding insulin, Pdx-1, and NeuroD. The latter step can include first, culturing the human BM cells for 24 to 48 hours to obtain adherent cells, and continuing to culture the adherent cells until they appear morphologically homogenous; and second, culturing the morphologically homogeneous cells for at least about 60 days in a medium with high levels of glucose at least until the cells express detectable levels of a pancreatic endocrine marker such as glucagon, insulin, or message (mRNA) encoding insulin, Pdx-1, or NeuroD. To facilitate growth, the cells can optionally be cultured for at least about 60 days in a medium including fibroblast growth factor (FGF), epidermal growth factor (EGF), and hepatocyte growth factor (HGF). To enhance differentiation, step (b) can further include culturing the cells for about 5 to 7 days in a low glucose medium supplemented with nicotinamide and/or exendin 4.

The cells of the invention can be contained in a liquid such as a tissue culture medium, and maintained at a temperature below 0° C., e.g., housed in a container in liquid nitrogen. The cells of the invention can also be those that have been introduced into a host animal subject, e.g., the human subject from which the HBMDS cells were obtained.

In another aspect, the invention features a method of reducing hyperglycemia in an animal subject. The method includes the step of transplanting into the animal subject an effective number of pancreatic marker-expressing cells differentiated from HBMDS cells by a method that includes the step of culturing the HBMDS cells in a high glucose containing medium.

Unless otherwise defined, all technical terms used herein have the same meaning as commonly understood by one of ordinary skill in the art to which this invention belongs.

Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the particular embodiments discussed below are illustrative only and not intended to be limiting.

### DETAILED DESCRIPTION

The invention provides insulin-producing cells isolated from an in vitro culture of BM-derived stem cells obtained from a human subject. It also provides methods of making and using such cells. The below described preferred embodiments illustrate adaptations of these cells and methods. Nonetheless, from the description of these embodiments, other aspects of the invention can be made and/or practiced.

### Biological Methods

Methods involving conventional biological, cell culture, immunological and molecular biological techniques are described herein. Such techniques are generally known in the art and are described in detail in methodology treatises. Cell culture techniques are generally known in the art and are described in detail in methodology treatises such as Culture of Animal Cells: A Manual of Basic Technique, 4th edition, by R. Ian Freshney, Wiley-Liss, Hoboken, N.J., 2000; and General Techniques of Cell Culture, by Maureen A. Harrison and Ian F. Rae, Cambridge University Press, Cambridge, UK, 1994. Immunological methods (e.g., preparation of antigen-specific antibodies, immunoprecipitation and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Molecular biological techniques are described in references such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Various techniques using polymerase chain reaction (PCR) are described, e.g., in Innis et al., PCR Protocols: A Guide to Methods and Applications, Academic Press: San Diego, 1990.

### Cells Differentiated to Express Pancreatic Endocrine Markers

The invention provides pancreatic endocrine marker-expressing cells that have been isolated from an in vitro culture of BM-derived stem cells obtained from a human subject. BM cells may be isolated from a subject by conventional methods, e.g., aspiration from the ileac crest of a human subject or from peripheral blood apheresis after mobilization of stem cells from BM by hematopoietic growth factors. See, Korbling and Anderlini, Blood 98, 2900-2908, 2001; Korbling et al., Blood 86, 2842-2848, 1995; Lane et al., Transfusion 39, 39-47, 1999; Thomas et al., Hamatol Bluttransfus 9, 86-95, 1970. Stem cells may be isolated from the BM by any suitable method, e.g., selecting for morphologically homogeneous adherent cells after several passages of a HBMDS cell culture. HBMDS cells can be differentiated into cells expressing pancreatic endocrine markers by the methods described below. In addition to expressing pancreatic endocrine markers, the differentiated cells of the invention might also exhibit functional characteristics of native pancreatic endocrine cells such as glucose-induced secretion of insulin.

To facilitate their handling the cells of the invention can be contained in a liquid, e.g., saline, a buffer at a physiological pH, a tissue culture medium, or serum. While the cells of the invention will often be maintained at body temperature (37° C.), for preservation, they might also be frozen at a temperature below 0° C. For example, the cells might be maintained in a −70° C. freezer or in liquid nitrogen. Cells frozen in this manner can be revived by thawing and placing in in vitro tissue culture according to conventional methods. The cells of the invention can also be contained in a host animal subject, e.g., introduced into the human subject from which the HBMDS cells were obtained, or introduced into a non-human animal that lacks the ability to reject the cells (e.g., a immuno-compromised animal)

### Method for Differentiating HBMDS Cells

The pancreatic endocrine marker-expressing cells of the invention can be obtained from HBMDS cells by culturing these cells under appropriate conditions. An important condition for effecting differentiation of HBMDS cells into pancreatic endocrine marker-expressing cells is culturing the HBMDS cells in a medium containing high glucose levels, e.g., greater than about 9 mM (e.g., 8.9, 9, 10, 11, 12, 13, 14, 15, 16, 17, 18, 19, 20, 21, 22, 23, 24, 25, 26, 27, 28, 29, and 30 mM) for at least about 60 days (e.g., 55, 60, 65, 70, 75, 80, 85, 90, 100 or more days). In the examples described below, culturing HBMDS cells for 60 days in a medium comprising a basal medium, serum or a serum substitute, and 23 mM glucose caused the cells to express detectable levels of glucagon, insulin, and mRNAs encoding insulin, Pdx-1, and NeuroD. The basal medium used in these experiments was RPMI 1640 although other basal media (e.g., DMEM, IMDM, and the like) might also be used. Serum such as fetal bovine serum (FBS) or a serum substitute is added to the medium to support cell growth.

Some versions of the method of the invention also include culturing the HBMDS cells for at least about 60 days in a medium that includes one or more growth factors such as FGF, EGF, and HGF; and/or culturing the cells for about 5 to 7 (e.g., 4, 5, 6, 7, 8, or 9) days in a low glucose (less than about 7.5 mM; e.g., 1, 2, 3, 4, 5, 5.5, 6, 7 mM) medium comprising nicotinamide and exendin 4. In some cases, β cell maturation factors might also be added. See Hellerstrom et al., Diabetes Supp. 2:89-93, 1991; and Buschard et al., Int. J. Exp. Diabetes Res. 1:1-8, 2000.

### Delivery of Differentiated Cells to a Host Subject

The cells made according to methods of the invention can be delivered to a subject, e.g., in an attempt to reverse a pancreatic endocrine hormone (e.g., insulin) deficiency in the subject. In this method, the differentiated pancreatic marker-expressing cells are transplanted into an animal (e.g., a mammal such as a rodent or a human patient suffering from a hormone deficiency). The hormone(s) secreted by the transplanted cells can then be released systemically to reduce or even reverse the deficiency.

In a particular application of this method, differentiated pancreatic insulin-producing cells are transplanted into a diabetic animal. Secretion of insulin by these cells should supply enough insulin to the animal to reduce or reverse its hyperglycemia or other symptoms of the disease. The effectiveness of particular protocols can be assessed using conventional clinical assays, e.g., determining the animal's insulin secretion response to a high glucose challenge, its ability to normalize circulating glucose levels, and its ability to maintain glucose homeostasis. For example, to test hormone-producing cells for the ability to reverse hyperglycemia in a mammal, NOD/SCID mice (Leung et al., Biol. Blood Marrow Transplant 5:107, 1999) or rats are induced to become diabetic by a series of intraperitoneal injections of streptozotocin, a β-cell toxin that causes a permanent destruction of pancreatic β cells. Prockop et al., Science 276:71-74, 1997; Prockop et al., Biochem. Soc. Trans. 28:341-345, 2000; and DiGirolamo et al., Br. J. Haematol. 107:275-281, 1999. Islet-like clusters or cells from one of the above-described cultures are transplanted to the renal subcapsular space, spleen, and liver locations in the diabetic animals to evaluate whether they can function as pancreatic β cells in vivo, e.g., produce insulin in response to a high glucose challenge. Islet-like clusters can be administered at a dose of approximately 1-100 clusters/gram body weight, and islet-like cells can be administered at a dose of approximately 1-2×10⁶ cells per animal (e.g., 1×10⁶ in the spleen and 1×10⁶ in the renal subcapsular space). For human subjects, administration of 5,000-10,000 clusters per kg is expected to be a suitable dose.

### Reversing Hyperglycemia

To reverse hyperglycemia in a subject, HBMDS cells are harvested from a donor animal, differentiated and matured into insulin-producing cells, and then transplanted into a host hyperglycemic animal subject. The cells can be transplanted into various locations in the recipient subject, e.g., liver, spleen, or renal subcapsular space. The recipient subject is then monitored for the ability to self-regulate blood glucose levels.

### Administration of Cells

The cells described above may be administered to animals including mammals (e.g., humans) in any suitable formulation. For example, pancreatic endocrine hormone-producing cells may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF.

The cells of the invention may be administered to animals by any conventional technique. The cells may be administered directly to a target site (e.g., a spleen or liver) by, for example, injection or surgical delivery to an internal or external target site, or by catheter to a site accessible by a blood vessel. The cells may be administered in a single bolus, multiple injections, or by intravenous continuous infusion.

### EXAMPLE 1

BM samples were obtained from human donors and transferred into test tubes containing ethylenediaminetetraacetic acid (EDTA). BM mononuclear cells were then isolated from the sample by lysis of all red blood cells and some of the late stage erythroid precursors with lysis buffer (8.29 g NH₄HCl, 1 g KHCO₃, 37 mg EDTA, distilled water to 1 L, pH 7.2), or by density centrifugation in a Ficoll-hypaque gradient to remove granulocytes followed by further lysis of red blood cells in a modified lysis buffer eliminating the EDTA. Isolated BM-derived cells were used to establish in vitro cell cultures in Medium A [Roswell Park Memorial Institute (RPMI) 1640 medium (Invitrogen Rockville, Md. cat# 11875-085, containing 5.5 mmol glucose) plus FBS (Hyclone, Logan Utah cat# SH30118.03) at 20%, 100 U penicillin (Invitrogen, Rockville, Md. cat#15240-062), 1000 U streptomycin (Invitrogen, Rockville, Md. cat#11860-038), and 1× insulin transferrin selenium (Invitrogen, Rockville, Md. cat# 41400-045)]. Cells were plated at 1×10⁵ cells ml⁻¹ in 6 well cell culture plates or at 1×10⁶ cells ml⁻¹ in T-25 flasks for 48 hours at 37° C. in a humidified 5% CO₂ incubator. After 48 hours the culture medium and non-adherent cells were removed. Medium A was then added back to the flasks and the cells were cultured for an additional two to four weeks until the spindle-shaped adherent cells reached 70-80% of confluence. The cells were then released from the surface with trypsin-EDTA (Sigma Chemicals) and re-plated at a 1:3 dilution under the same culture conditions. This re-plating was repeated numerous times until the cells appeared morphologically homogenous. To induce differentiation, HBMDS cells were released from the surface with trypsin-EDTA (Sigma Chemicals) and placed into culture flask containing Medium B [RPMI 1640 containing 5.5 mM glucose; 10% FBS; an additional 17.5 mM glucose; plus or minus various growth factors including basic FGF (bFGF; 1 ng/ml, Sigma, St. Louis, Mo. cat#F0291) and EGF (EGF, 10 ng/ml, Sigma, St. Louis, Mo. cat#H1404); 100 U penicillin; 1000 U streptomycin; and fresh glutamine]. Cell differentiation was monitored by observing morphologic features such as cluster formation and by molecular biological techniques such as RT-PCR and immunocytochemical staining. To promote maturation of BM-derived pancreatic endocrine-like cell precursors after expansion and differentiation, the cells were switched to Medium C [RPMI 1640 with a low glucose concentration (5.5 mM), supplemented with nicotinamide (10 mM, sigma), and ITS premix (5 μg/ml insulin, 5 μg/ml transferrin, and 5 μg/ml selenium; Invitrogen, Rockville, Md. cat# 41400-045) and 5% FBS]. Other β cell maturation factors (such as HGF, TGF, IGF, EGF, activin A, and betacellulin) were also added to the cell culture media to maximize cell maturation. This step facilitated cluster formations both in number and in mass. The three-dimensional clusters were morphologically similar to the islets.

To determine whether the islet-like clusters appearing in the BM cell cultures trans-differentiated to endocrine-hormone expressing cells, gene expression of endocrine cell differentiation markers and hormones was measured using RT-PCR. The mRNA used in this analysis was purified from total RNA using oligo-dT columns and cDNA was synthesized from the mRNA using random hexamer primers. RT-PCR was performed to detect islet 1, neurogenin 3 and insulin. All PCR products were sequenced and each sequence was compared to the published sequences of each gene. Undifferentiated BM stem cells cultured two-months after isolation expressed no detectable levels of islet 1, neurogenin 3 or insulin. The BM stem cell cultures grown in a high concentration of glucose that formed islet-like clusters expressed pancreatic endocrine differentiation markers (i.e., islet 1 and neurogenin 3) and endocrine hormones (i.e., insulin).

HBMDS cells after 6 months of culturing in the RPMI 1640 medium containing 10% fetal bovine serum began to form small clusters after plating on fibronectin-coated plates. These cells were further induced to differentiate under high glucose conditions (e.g., 23 mM glucose). To determine if the differentiated BM-derived cells actually synthesized endocrine hormone proteins, cells were first detached by 0.25% of trypsin-EDTA, then incubated in Medium C in a 10 mL test tube in an incubator for two hours before embedding in a paraffin cell block. The presence of the endocrine cell hormones insulin and glucagon in trans-differentiated BM-derived cells was detected by immunocytochemical staining. The paraffin block sections were stained with either hematoxylin-eosin (H&E) stains for morphologic evaluation or probed with the primary antibodies against the endocrine hormones insulin (polyclonal guinea pig anti-rat, DAKO, Carpinteria, Calif.) and glucagon (DAKO, Carpinteria, Calif.). Antibodies to human albumin and cytokeratin CK19 were used as negative controls. Human pancreas was used as a positive control. The results showed pancreatic endocrine hormone (insulin and glucagon) production after 2 weeks of continued growth under high glucose conditions. The majority of cells stained strongly for glucagon (90% of cells) and 5 to 10% of the cells stained positive for insulin. The differentiated cells did not stain for albumin or CD19 (negative controls).

### EXAMPLE 2

Methods

BM was obtained from 20 healthy donors (age 2 to 50 years, mean 25±3 years) according to guidelines from the University of Florida Institutional Review Board on the use of human subjects in research. Human BM mononuclear cells were obtained by Ficoll-Plaque density gradient centrifugation (Sigma Chemical, St. Louis, Mo.) to remove mature leukocytes and red blood cells.

The rat INS-1 cell line (clone 832/13), a cell line capable of insulin release in response of glucose stimulation, was a generous gift from Dr. Christopher B. Newgard (Duke University, Durham, N.C.). This cell line was derived from stable transfection of a plasmid containing the human proinsulin gene and expresses and processes both rat and human insulin. The cells were maintained in RPMI 1640 medium with 11.1 mM D-glucose supplemented with 10% FBS as described in Hohmeier et al., Diabetes 49:424-430, 2000. This cell line was used as a positive control for studies of insulin content and insulin release.

Antibodies against CD45, CD34, CD117, CD38, CD64, CD14, CD13, CD33, CD11b, CD56, CD44, CD90, CD49b, CD19, CD20, CD2, CD5, CD4, CD8, CD3, CD7, HLA-DR, Class I HLA, and β2 microglobulin were purchased from Becton Dickinson Biosciences (San Jose, Calif.). Rabbit anti-insulin polyclonal IgG (Santa Cruz Biotechnology, Santa Cruz, Calif.) for immunogold study, polyclonal guinea pig anti-insulin and rabbit anti-rat glucagon (DAKO Corporation, Carpinteria, Calif.), rabbit anti-rat-C-peptide antibody (LINCO Research, St. Charles, Mo.), anti-rabbit IgG and Guinea pig serum, Cy3-coupled anti-guinea pig IgG (DAKO Corporation), were obtained and utilized for immunocytochemistry.

For these studies, a variety of reagents were utilized as described, FGF (FGF) from Sigma, St. Louis, Mo., EGF (EGF), HGF (HGF), vascular endothelial growth factor (VEGF), all from Peprotech, Rocky Hill, N.J., nicotinamide (10 mM) and exendin 4 (10 nM) from Sigma and fetal calf serum (FCS) from HyClone, Logan, Utah.

Culture of HBMDS cells. The human BM mononuclear cells were plated in RPMI 1640 plus 20% FBS for 24 to 48 hours (37° C./5% CO₂). Unattached cells were removed by washing twice with medium and the adherent cells grown in the same medium until 70 to 80% confluence before passage. Following 3 to 4 passages, HBMDS cells became morphologically homogeneous. At this stage, single cell-derived HBMDS cell lines were cloned using a cloning cylinder (Fisher Scientific, Pittsburgh, Pa.). The selected cells were expanded and used for phenotypic characterization and for in vitro differentiation. Studies of the in vitro differentiation and characterization of the HBMDS cells utilized a single cell-derived clone from BM of a 10-year-old donor without diabetes or hematological disorders.

Flow cytometric analysis. Uncloned HBMDS cells from 10 donors and the cloned cell lines were stained with either fluorescein isothiocyanate-conjugated (FITC) or phycoerythrin-conjugated (PE) antibodies (Becton Dickinson) against cell surface antigens following manufacturer's instructions to obtain the phenotype of the HBMDS cells under in vitro culture conditions. These included antibodies against CD45, CD34, CD117, CD38, CD64, CD14, CD13, CD33, CD11b, CD56, CD44, CD90, CD49b, CD19, CD20, CD2, CD5, CD4, CD8, CD3, CD7, MHC-II, MHC-I, and beta 2 microglobulin. Approximately 3×10⁵ undifferentiated HBMDS cells were stained as described in Li et al., Blood 101:1977-1980, 2003. The data were analyzed by flow cytometry using FCS express 2 software (De-Novo software, Ontario, Canada). Controls utilized FITC- and PE-conjugated isotype-matched immunoglobulins. Samples were analyzed in triplicate. For every sample, 3×10⁴ cells were acquired.

The developmental plasticity of HBMDS cells was examined by testing the cells' ability to differentiate into endothelial cells after 14 days in an in vitro culture containing 10 ng/ml VEGF. The endothelial cell phenotype was examined by detecting surface expression of various vascular antigens including CD31, and von Willebrand factor (vWF), (DAKO Corporation) by flow cytometric analysis.

Differentiation cultures. In order to induce the HBMDS cells to differentiate into cells with a pancreatic endocrine phenotype, the cloned cells were cultured in a basic medium composed of RPMI 1640 medium with the addition of 17.5 mM glucose (23 mM final concentration) and 10% FCS for two to four months. To further expand and promote cellular differentiation, the cells were cultured in medium plus 1 ng/ml FGF, 10 ng/ml EGF, and 10 ng/ml HGF for an additional two months. To promote cellular maturation, the cells were cultured for 5 to 7 days in RPMI 1640 medium with a low glucose concentration (5.0 mM), a lower concentration of FCS (5%), plus nicotinamide (10 mM) and exendin 4 (10 nM). The low glucose and low FCS medium without growth factors inhibited cell proliferation, promoted cell differentiation and maturation, and increased the cells' sensitivity to glucose stimulation.

RT-PCR. Total RNA was prepared from HBMDS cell cultures at various stages, including two to four weeks of low glucose culture and two to four months of high glucose culture using TRIzol reagent. Transcriptional gene expression related to pancreatic organ genesis from these cultures was determined by RT-PCR according to a published protocol (Yang, et al., Proc. Natl. Acad. Sci. U.S.A. 99:8078-8083, 2002) with minor modifications. The forward and reverse primers of each PCR set were designed to be located in different exons based on sequences obtained from GenBank. PCR products were separated by electrophoresis in 2.5% agarose gel, and the sequence of each PCR product confirmed by Big-Dye DNA sequence analysis using the ABI-377 sequencer following the manufacturer's protocols.

Immunocytochemistry and immunofluorescence. Sterile microscope slide cover slips were coated with fibronectin and placed in tissue culture plate wells. Differentiated HBMDS cells were fixed with 4% paraformaldehyde for 30 min at room temperature, and each slide frozen at −70° C. until assay. Immunocytochemistry was performed with polyclonal guinea pig anti-insulin (1:500) (DAKO Corporation) and guinea pig anti-rat C-peptide antibody (1:100) (LINCO Research) for 1 hour. After washing three times, the cells were incubated with Cy3-coupled anti-guinea pig (1:1000) secondary antibodies (Research Diagnostics Inc, Flanders, N.J.) for 30 min. Guinea pig serum was used as negative control. Cells were examined by fluorescence microscopy (Olympus B×51) as well as by confocal fluorescence microscopy. In addition, cytospin slides from differentiated HBMDS cells were prepared, air-dried, and kept frozen at −70° C. until assayed for insulin and c-peptide protein expression. A cellblock was made from differentiated HBMDS cells by first releasing the cells with 0.25% trypsin EDTA, and then incubating the cells in the culture medium for two hours in a cell culture incubator before the paraffin cellblock was made. The cellblock was used for H&E staining and immunohistochemistry for insulin and glucagon. Positive staining was visualized with Vector Blue (LSAB Kit, DAKO) for insulin and alkaline phosphatase and DAB for glucagon. The cell nuclei were counter-stained with Nuclear Fast red and

Human insulin ELISA. Differentiated HBMDS cells were cultured in the presence or absence of 10-mM nicotinamide, or exendin 4, or both for 5-7 days in RPMI 1640 containing 5% FBS, and 5.5 mM glucose after the cells were confirmed to express insulin genes by RT-PCR. The cells were switched to serum-free medium containing 0.5% bovine serum albumin (BSA) for 12 hrs, washed twice with PBS, then stimulated by the addition of 17.5 mM additional glucose (final concentration of 23 mM) for various times. The culture media were collected and frozen at −70° C. until assayed for insulin release. The serum-free culture medium containing 0.5% BSA was used as a control for secreted insulin measurements. Insulin release was detected by using a human insulin ELISA kit (ALPCO Diagnostics, Windham, N.H.) with sensitivity of 0.15 μU/ml following the manufacturer's protocols. This assay does not detect proinsulin.

Deconvolution microscopy. Cells were stained with Cy3-conjugated secondary antibodies after they were incubated with antibodies specific for insulin or C-peptide. The nuclei were counter-stained with DAPI and the cells were subjected to analysis using deconvolution microscopy on a Delta Vision Olympus OMT inverted fluorescent microscope and Unix software system. The images depict 3-dimensional projections of 25-0.2 micron optical slices through the cell, center focused on the DAPI stained chromatin in the nuclei. All images were scale-adjusted, including images of staining with non-specific isotype antibody conjugates as negative controls.

Electron microscopy. After washing with PBS, cells were preserved in 1% glutaraldehyde, 2% paraformaldehyde in PBS for 1 hour, washed with PBS, harvested, washed with 0.1 M Na cacodylate, then post-fixed with 2% OsO₄ in 0.1 M Na cacodylate containing 1 mM CaCl₂. The samples were embedded in TAAB epoxy resin (Marivac, Ltd., Halifax, Nova Scotia). Ultrathin sections were counterstained with uranyl acetate and lead citrate, and then viewed using a Zeiss EM-10A transmission electron microscope. For immunogold localization of insulin, the cells were embedded in Lowicryl K4M resin (Electron Microscopy Sciences, Fort Washington, Pa.). Ultrathin sections were mounted on formvar/carbon-coated nickel grids and subjected to the immunogold labeling procedure. Briefly, the sections were blocked with 5% BSA, 5% normal goat serum, and 0.1% cold water fish skin gelatin in PBS, rinsed, and then incubated overnight at 4° C. in the rabbit anti-insulin antibody (Santa Cruz Biotechnology, Santa Cruz, Calif.) diluted 1:50 in PBS containing 0.2% acetylated BSA (Aurion BSAc, Electron Microscopy Sciences) and 10 mM NaN₃. After washing, the samples were incubated for 1.5 hours at room temperature with the secondary goat anti-rabbit IgG antibody conjugated to 0.8 nm colloidal gold particles (Aurion EM Grade Ultra Small, Electron Microscopy Sciences), then washed, treated with 1.25% glutaraldehyde in PBS, and washed again. The gold particles were silver-enhanced for 45 minutes at room temperature (Aurion R-Gent SE EM, Electron Microscopy Sciences). The samples were counterstained using uranyl acetate and lead citrate, then viewed using a Zeiss EM-10A transmission electron microscope.

Results

Characterization of the HBMDS cells. The HBMDS cells were enumerated at each passage utilizing a haemocytometer. After 3-4 passages, the cells at 106 population doublings were labeled with FITC-, PE-, or Per-CP-coupled antibodies against CD45, CD34, CD117, CD38, CD64, CD14, CD13, CD33, CD11b, CD56, CD44, CD90, CD49b, CD19, CD20, CD2, CD5, CD4, CD8, CD3, CD7, HLA-DR, Class I HLA, and β2 microglobulin. Isotype-matched immunoglobulin served as control antibodies. Cells were analyzed by flow cytometry. The phenotype of cultured mixed and cloned HBMDS cells were identical; both were negative for leukocyte common antigen (CD45), hematopoietic stem cell markers (CD34, CD38, and CD117), monocytic markers (CD64 and CD14), myeloid lineage markers (CD33, CD11b), a natural killer cell marker (CD56), T-cell markers (CD2, CD5, CD3, CD5, CD4, CD8, and CD7), and B-cell markers (CD19, and CD20). These cells also do not express class II HLA-DR. However the cells weakly expressed CD49b and CD44, and strongly expressed CD90, CD13, beta-2-microglobulin and class I HLA. Thus, the morphology and phenotype were similar after 40 to more than 106 population doublings. These cells exhibited the capacity of cell renewal and differentiation into endothelial-like cells after 14 days of incubation with VEGF. The cells were stored in liquid nitrogen and, when re-started, the morphology and immunophenotype remained unchanged.

In vitro differentiation of HBMDS cells. To induce cell differentiation, the cloned HBMDS cells were switched into RPMI 1640 medium containing 10% FCS, high glucose (23 mM), and with or without various growth factors as described above. After two to four months of in vitro induction, the cells began to form a three-dimensional cluster. To promote maturation of these cells into insulin-secreting cells, the cells were switched to the medium containing 5% FCS with 10 mM nicotinamide, exendin 4, and low concentration of glucose (5.5 mM) after the expansion of the differentiated pancreatic endocrine precursor cells. This step increased both the number and mass of clusters that formed, and also increased the sensitivity of glucose responsiveness.

Gene expression of HBMDS cells and differentiated HBMDS cells. To determine if the islet-like clusters that appeared in the cell cultures were made up of endocrine-hormone expressing cells, the gene expression of endocrine cell differentiation markers and hormones was measured using RT-PCR at various stages of the in vitro differentiation process. All PCR products were sequenced and each sequence was compared to the published sequence of the gene. Undifferentiated HBMDS cells, cultured for eight weeks after isolation, expressed no detectable levels of islet-1, neurogenin 3 (Ngn-3), or insulin. In contrast, the differentiated HBMDS cells began to express pancreatic endocrine differentiation markers involved in islet cell development (islet 1, Ngn 3) at two or four weeks of differentiation culture but there was no detectable expression of the insulin gene at this time. Insulin gene expression became detectable at eight weeks of differentiation culture. Gene expression of insulin, Pdx-1, NeuroD was observed at 12 weeks of differentiation culture, and persisted throughout the time of observation, whereas the expression of the islet-1 and ngn-3 genes became undetectable at 12 weeks of culture.

Endocrine hormone synthesis by differentiated HBMDS cells. To determine if the differentiated HBMDS cells actually synthesized the endocrine hormone proteins, sections of the cells from a paraffin cellblock were stained with either H&E for morphologic evaluation or probed with the primary antibodies against the endocrine hormones insulin and glucagon. Antibodies to human albumin and cytokeratin CK19 were used as negative control. Human pancreas was used as positive control. Expression of insulin and glucagon was observed in the cytoplasm of HBMDS cells after 10 weeks of growing in a high glucose medium. The majority of cells were stained strongly for cytoplasmic glucagon, but only 5 to 10% of cells were stained positive for insulin. The differentiated cells did not stain for albumin or CD 19 (negative controls).

Immunofluorescence analysis of insulin and C-peptide synthesis. To evaluate the continuing cell expansion, differentiation, and maturation, the differentiated cells were cultured for additional 4 weeks in the presence of growth factors, nicotinamide, and exendin 4, as described above, and subsequently stained with anti-insulin and anti-C-peptide antibodies and visualized under a fluorescence microscope. This culture step resulted in a marked increase in the percentage of the insulin-producing cells (20% of cells had strong staining and 50% of cells had weak cytoplasmic staining for insulin). Strong C-peptide cytoplasmic staining was detected in 20 to 30% of the examined cells. The rat insulinoma cell line INS-1 cells were used as a positive control for insulin and c-peptide immunostaining.

In order to study the distribution of insulin granules, deconvolution microscopy combined with immuno-labeling using anti-insulin and anti-c-peptide antibodies was used to compare the insulin granule and c-peptide distribution in the in-vitro-differentiated HBMDS cells with that in INS-1 insulinoma cells. The results showed that the insulin granules in the differentiated cells were arranged in a polarized fashion with most of the granules being situated within one side of the cell similar to the location in INS-1 cells. This pattern is consistent with insulin being released in a physiologic response to glucose stimulation.

Insulin release in response to glucose stimulation. To determine whether the differentiated HBMDS cells are responsive to a glucose challenge, the time course of insulin release from the differentiated HBMDS cells with various culture conditions was measured using a human insulin ELISA kit. In order to increase the sensitivity of the cells to a high glucose challenge, the cells were switched to low serum, low glucose medium plus either exendin 4, nicotinamide, or both for five days. The cells then were washed twice with PBS and switched to serum-free low glucose medium containing 0.5% BSA overnight, then stimulated by the addition of 23 mM glucose for various times up to eight hours. The amount of insulin released into the cell culture media was then quantified. All samples were in triplicates. The results showed that peak insulin release occurred two hours after the glucose challenge in the culture pretreated with exendin 4. Insulin release in cells pretreated with both nicotinamide and exendin 4 occurred much earlier and peaked within a few minutes in response to a glucose challenge and returned to a lower level at 1 hr. Moreover, the intensity of insulin release after pretreatment with both nicotinamide and exendin 4 was much stronger than after exendin 4 treatment alone. Surprisingly, there was no detectable insulin release when the cells were treated with nicotinamide alone.

Ultrastructure analysis by electron microscopy and immunogold labeling. The differentiated HBMDS cells were cultured for 7 days after nicotinamide treatment to reach a higher degree of maturation. At this stage, the cells produced and secreted insulin. In order to demonstrate the characteristics of insulin-containing secretory granules, electron microscopy combined with immunogold labeling with anti-insulin antibodies was performed to compare the in vitro differentiated HBMDS cells with INS-1 insulinoma cells. The results showed numerous globular structures having an electron-dense core surrounded with a pale halo area were present in both INS-1 and the differentiated HBMDS cells. The immunogold labeling of the differentiated HBMDS cells confirmed that the globular structures contained insulin visualized by the presence of dark electron-dense particles within the granules, although much less than observed in INS-1 insulinoma cells (which over-express insulin).

### EXAMPLE 3

Transplantation of human pancreatic-marker expressing cells differentiated from HBMDS cells reduces hyperglycemia. NOD-SCID mice received daily intraperitoneal injections of streptozotocin (50 mg/kg body weight) for five days to induce diabetes. Blood glucose levels were monitored daily using an Accu-CHEK glucose detector (Roche Diagnostics, Indianapolis, Ind.). Within 12 days after the injections, all mice became hyperglycemic with blood glucose levels >350 mg/dl. The differentiated HBMDS cells were transplanted to both the renal subcapsular space (1×10⁶/mouse) and the distal tip of the spleen (1×10⁶/mouse) of six mice when the blood glucose levels of the diabetic mice exceeded 350 mg/dl. Six control mice received sham surgery without implants. The blood glucose levels were monitored every 4 days following transplantation for 56 days. The animals were not food deprived. About eight days after transplantation, those mice receiving the differentiated cells exhibited a substantial decrease in glucose levels (about 50%). This reduction was maintained over the entire monitoring period. In comparison, control mice did not exhibit a substantial decrease in glucose levels. In addition, two mice from the experiment group underwent splenectomy after transplant. This procedure resulted in an increase in blood glucose level, indicating that the transplanted cells contributed to the reduced blood glucose levels.

### Other Embodiments

It is to be understood that while the invention has been described in conjunction with the detailed description thereof, the foregoing description is intended to illustrate and not limit the scope of the invention, which is defined by the scope of the appended claims. For example, although the above description relates to human cells, various aspects of the invention might also be applied to cells from other animals (e.g., mammals such as mice, rats, cows, sheep, monkeys, apes, horses, goats, cats, dogs, pigs, etc.) by making appropriate modifications to the described methods. Other aspects, advantages, and modifications are within the scope of the following claims.

What is claimed is:

1. An insulin-producing cell isolated from an in vitro culture of bone marrow cells obtained from a human subject.

2. A cell that expresses detectable levels of glucagon, insulin, and mRNAs encoding insulin, Pdx-1, and NeuroD, the cell being isolated from a culture of human bone marrow cells prepared by a method comprising the steps of:
(a) obtaining human bone marrow mononuclear cells from a human subject; and
(b) culturing the obtained human bone marrow mononuclear cells under conditions that cause the cells to express detectable levels of glucagon, insulin, and mRNAs encoding insulin, Pdx-1, and NeuroD.

3. The cell of claim 2, wherein the step (b) of culturing the human bone marrow mononuclear cells under conditions that cause the cells to express detectable levels of glucagon, insulin, and mRNAs encoding insulin, Pdx-1, and NeuroD comprises:
(i) first, culturing the human bone marrow mononuclear cells in a tissue culture container for about 24 to 48 hours to obtain cells that adhere to the container (adherent cells), and continuing to culture the adherent cells until they become morphologically homogenous; and
(ii) second, culturing the morphologically homogeneous cells for at least about 60 days in a medium comprising about 9 to 30 mM glucose at least until the cells express detectable levels of glucagon, insulin, and mRNAs encoding insulin, Pdx-1, and NeuroD.

4. The method of claim 3, wherein the medium comprises about 23 mM glucose.

5. The cell of claim 3, wherein step (ii) further comprises culturing the cells for at least about 60 days in a medium comprising at least one growth factor selected from the group consisting of: FGF, EGF, and HGF.

6. The cell of claim 3, wherein the step (b) of culturing the human bone marrow mononuclear cells under conditions that cause the cultured cells to express detectable levels of glucagon, insulin, and mRNAs encoding insulin, Pdx-1, and NeuroD further comprises:
(iii) third, culturing the cells for about 5 to 7 days in a medium comprising less than about 7.5 mM glucose and at least one agent selected from the group consisting of nicotinamide and exendin 4.

7. The cell of claim 2, wherein the cell secretes insulin when placed in a medium comprising about 23 mM glucose.

8. The cell of claim 1, wherein the cell is comprised in a liquid.

9. The cell of claim 8, wherein the liquid is a tissue culture medium.

10. The cell of claim 2, wherein the cell is comprised in a liquid.

11. The cell of claim 10, wherein the liquid is a tissue culture medium.

12. The cell of claim 1, wherein the cell is at a temperature below 0° C.

13. The cell of claim 12, wherein the cell is housed in a container in liquid nitrogen.

14. The cell of claim 2, wherein the cell is at a temperature below 0° C.

15. The cell of claim 14, wherein the cell is housed in a container in liquid nitrogen.

16. The cell of claim 1, wherein the cell has been introduced into a host animal subject.

17. The cell of claim 2, wherein the cell has been introduced into a host animal subject.

18. The cell of claim 16, wherein the cell host animal subject is the human subject from which the human bone marrow cells were obtained.

19. The cell of claim 17, wherein the cell host animal subject is the human subject from which the human bone marrow mononuclear cells were obtained.

20. A method for making cells that express detectable levels of glucagon, insulin, and mRNAs encoding insulin, Pdx-1, and NeuroD, the method comprising the steps of:
(a) isolating human bone marrow mononuclear cells from a human subject; and
(b) culturing the isolated human bone marrow cells under conditions that cause the cultured cells to express detectable levels of glucagon, insulin, and mRNAs encoding insulin, Pdx-1, and NeuroD.

21. The method of claim 20, wherein the step (b) of culturing the isolated human bone marrow cells under conditions that cause the cultured cells to express detectable levels of glucagon, insulin, and mRNAs encoding insulin, Pdx-1, and NeuroD comprises:
(i) first, culturing the human bone marrow mononuclear cells in a tissue culture container for about 24 to 48 hours to obtain cells that adhere to the container (adherent cells), and continuing to culture the adherent cells until they become morphologically homogenous; and
(ii) second, culturing the morphologically homogeneous cells for at least about 60 days in a medium comprising about 9 to 30 mM glucose at least until the cells express detectable levels of glucagon, insulin, and mRNAs encoding insulin, Pdx-1, and NeuroD.

22. The method of claim 21, wherein step (ii) further comprises culturing the cells for at least about 60 days in a medium comprising at least one growth factor selected from the group consisting of: FGF, EGF, and HGF.

23. The method of claim 21, wherein the step (b) of culturing the isolated human bone marrow cells further comprises:
(iii) third, culturing the cells for about 5 to 7 days in a medium comprising less than about 7.5 mM glucose and at least one agent selected from the group consisting of nicotinamide and exendin 4.

24. A method of reducing hyperglycemia in an animal subject, the method comprising transplanting into the animal subject an effective number of pancreatic marker-expressing cells differentiated from human bone marrow-derived stem cells by a method comprising the step of culturing the human bone marrow-derived stem cells in a high glucose containing medium.
